(19) United States
(12) Patent Application Publication
(10) Pub. No.: US 2007/0117123 A1
(43) Pub. Date: May 24, 2007

(54) SALIVARY PROTEIN AND RNA FOR BREAST CANCER DETECTION

(75) Inventors: David T. W. Wong, Beverly Hills, CA (US); Mai Nguyen Brooks, Westlake Village, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California

(21) Appl. No.: 11/531,967

(22) Filed: Sep. 14, 2006

Related U.S. Application Data
(60) Provisional application No. 60/717,154, filed on Sep. 14, 2005.

(51) Int. Cl. C12Q 1/68; G01N 33/574; C12P 19/34

(52) U.S. Cl. 435/6; 435/7.23; 435/91.2

(57) ABSTRACT

The present invention provides for the first time the identification of salivary protein and RNA factors that are elevated in breast cancer. The present invention therefore provides methods of diagnosing and providing a prognosis for breast cancer, by examining cancer antigens (protein and RNA) in saliva.

### CROSS-REFERENCES TO RELATED APPLICATIONS

The present application claims priority to provisional application U.S. Ser. No. 60/717,154, filed Sep. 14, 2005, herein incorporated by reference in its entirety.

### STATEMENT AS TO RIGHTS TO INVENTIONS MADE UNDER FEDERALLY SPONSORED RESEARCH OR DEVELOPMENT

This invention was made with Government support under NIH/NIDCR Grant Nos. UO1 DE 15018 and UO1 DE 16275. The Government has certain rights in this invention.

### BACKGROUND OF THE INVENTION

Breast cancer is the most common form of cancer and the second leading cause of cancer deaths in American women. In 2004, approximately 215,990 women and 1,500 men were diagnosed with invasive breast cancer, and 40,580 were estimated to die of this disease (1). Early detection has been credited for a small decrease in age-adjusted breast cancer mortality. However, the current standard diagnostic/screening tests for breast cancer including physical exams and mammograms are not perfect, and thus there is much active research in developing novel methods to improve early detection.

Serum tumor markers, such as CEA (carcinoembryonic antigen) and CA15-3 or CA27-29, are used in current clinical practice to assess widespread disease or to detect recurrent breast cancer, not to detect new breast cancer (2). Many researchers are using a number of new technologies, such as proteomics or DNA/RNA arrays, to discover novel markers in the blood (3-4). While saliva is a source of easily accessible bodily fluids, there has been very little effort to study salivary fluid. We hypothesized that a profile of angiogenic and tumor markers in saliva could be complementary to the current methods used for breast cancer diagnosis. In this study, we set out to determine whether the levels of certain growth/tumor marker(s) is/are correlated with breast cancer. We studied VEGF (vascular endothelial growth factor) and EGF (epidermal growth factor) because they are potent angiogenic factors, with successful targeted therapeutic agents either already approved by the FDA (Avastin, Tarceva, etc.) or in ongoing clinical trials (5-6). We also measured CEA, a well established serum tumor marker for breast cancer. We observed that the levels of the above proteins in the saliva are elevated in breast cancer patients, in comparison to normal controls.

### BRIEF SUMMARY OF THE INVENTION

In one aspect, the present invention provides a method of diagnosing breast cancer in a subject, the method comprising the steps of: (a) contacting a saliva sample from the subject with a reagent that specifically binds to a marker selected from the group consisting of VEGF, CEA, and EGF nucleic acid and VEGF, CEA, and EGF polypeptide; and (b) determining whether or not the marker is overexpressed in the sample; thereby providing a diagnosis for breast cancer.

In another aspect, the present invention provides a method of providing a prognosis for a breast cancer, the method comprising the steps of: (a) contacting a saliva sample from the subject with a reagent that specifically binds to a marker selected from the group consisting of VEGF, CEA, and EGF nucleic acid and VEGF, CEA, and EGF polypeptide; and (b) determining whether or not the marker is overexpressed in the sample; thereby providing a prognosis for breast cancer.

In another aspect, the present invention provides a method of monitoring the efficacy of a treatment for a breast cancer, the method comprising the steps of: (a) analyzing a saliva sample from the subject with an assay that specifically detects a marker selected from the group consisting of VEGF, CEA, and EGF nucleic acid and VEGF, CEA, and EGF polypeptide; and (b) determining whether or not the marker is overexpressed in the sample; thereby monitoring the efficacy of a treatment for breast cancer.

In one embodiment, the assay comprises a reagent that binds to a protein. In another embodiment, the assay comprise a reagent that is an antibody. In another embodiment, the reagent is a monoclonal antibody.

In one embodiment, the reagent binds to a nucleic acid. In another embodiment, the reagent is a nucleic acid. In another embodiment, the reagent is an oligonucleotide. In another embodiment, the reagent is an RT-PCR primer set.

In one embodiment, the assay detects protein and is ELISA, Western blotting, flow cytometry, immunofluorescence, immunohistochemistry, or mass spectroscopy. In another embodiment, the assay detects nucleic acid and is mass spectroscopy, PCR, microarray hybridization, thermal cycle sequencing, capillary array sequencing, or solid phase sequencing.

In one embodiment, comprising the step of analyzing a saliva sample from the subject with an assay that specifically detects VEGF and EGF and determining whether or not VEGF and EGF protein are overexpressed in the sample.

### BRIEF DESCRIPTION OF THE DRAWINGS

FIG. 1: ROC curve of salivary VEGF and EGF values in specimens from control subjects and breast cancer patients. Area under the ROC curve 84%.

### DETAILED DESCRIPTION OF THE INVENTION

Introduction

We have now demonstrated the diagnostic/prognostic significance of VEGF, CEA, EGF and combinations thereof, such as VEGF plus EGF, VEGF plus CEA, CEA plus EGF, and VEGF, CEA, and EGF in saliva samples for human breast cancer. Either protein or RNA can be detected in saliva. Detection of these and other cancer antigens in saliva is therefore useful for diagnosis and prognosis of breast cancer as well as other cancers. In terms of early diagnosis, cancer antigens (proteins and RNA encoding the protein) in saliva can be examined by techniques such as ELISA, RT-PCR or mass spectroscopy, alone or in combination with other markers such as HER2/Neu, CA15-3, CA27-29. Any specific probe can be used for detection, such as an antibody, a receptor, a ligand, RT-PCR etc. Mass spectroscopy can also be used for protein detection. Thus, the present invention can be used alone or as a complement to traditional antigen analysis to enhance the diagnosis of breast and other cancers.

The present invention also provides the diagnostic/prognostic significance of VEGF, CEA, EGF and combinations thereof, such as VEGF plus EGF, VEGF plus CEA, CEA plus EGF, and VEGF, CEA, and EGF in serum samples for human breast cancer. Either protein or RNA can be detected in saliva. Detection of these and other cancer antigens in saliva is therefore useful for diagnosis and prognosis of breast cancer as well as other cancers. In terms of early diagnosis, cancer antigens (proteins and RNA encoding the protein) in saliva can be examined by techniques such as ELISA, RT-PCR or mass spectroscopy, alone or in combination with other markers such as HER2/Neu, CA15-3, CA27-29. Any specific probe can be used for detection, such as an antibody, a receptor, a ligand, RT-PCR etc. Mass spectroscopy can also be used for protein detection. Thus, the present invention can be used alone or as a complement to traditional antigen analysis to enhance the diagnosis of breast and other cancers.

Generally, the methods find particular use in diagnosing or providing a prognosis for breast cancer, as well as other cancers. While saliva is a source of easily accessible bodily fluids, there has been little effort to study its value in cancer diagnosis. We hypothesized that certain proteins and RNA would be elevated in the saliva of patients with breast cancer. In addition, elevation of proteins and RNA in patients with breast cancer is associated with a poor prognosis, including disease free survival, overall survival, and metastatic cancer.

There were 49 healthy individuals and 49 breast cancer patients in our study. The levels of vascular endothelial growth factor (VEGF), epidermal growth factor (EGF), and carcinoembryonic antigen (CEA) in the saliva were measured with enzyme linked immunosorbent assay (ELISA). The Wilcoxon test and multiple linear regression model were used to assess the association of breast cancer and salivary peptides, and the predictive power was analyzed by the receiver operating characteristic (ROC) curve.

The salivary fluid protein levels were significantly elevated in cancer patients as follows: 1) VEGF: 3.7±1.6 in cancer versus 2.1+1.2 ng/ml in control (p<0.0001); 2) EGF: 3.7±1.7 versus 2.1±1.3 ng/ml (p<0.0001); and 3) CEA: 83±31 versus 66.1±27.1 ng/ml (p=0.0106). The areas under the ROC curve (AUC) were 80%, 77%, and 65%, respectively. The best prediction in this study was from the combination of salivary VEGF and EGF with a sensitivity of 83%, specificity of 74%, and AUC 84%.

Definitions

“VEGF,” “CEA,” and “EGF” refer to nucleic acids, e.g., gene, pre-mRNA, mRNA, and polypeptides, polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to a polypeptide encoded by a referenced nucleic acid or an amino acid sequence described herein; (2) specifically bind to antibodies, e.g., polyclonal antibodies, raised against an immunogen comprising a referenced amino acid sequence, immunogenic fragments thereof, and conservatively modified variants thereof; (3) specifically hybridize under stringent hybridization conditions to a nucleic acid encoding a referenced amino acid sequence, and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a reference nucleic acid sequence. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules. The protein sequence for VEGF is provided, for example, by Accession No. NP_{—}001020537; The protein sequence for EGF is provided, for example, by Accession No. NP_{—}958439. The protein sequence for CEA is provided, for example, by Accession No. CAA44076. Truncated and alternatively spliced forms of these antigens are included in the definition.

“Cancer” refers to human cancers and carcinomas, sarcomas, adenocarcinomas, lymphomas, leukemias, etc., including solid and lymphoid cancers, kidney, breast, lung, kidney, bladder, colon, ovarian, prostate, pancreas, stomach, brain, head and neck, skin, uterine, testicular, esophagus, and liver cancer, including hepatocarcinoma, lymphoma, including non-Hodgkin's lymphomas (e.g., Burkitt's, Small Cell, and Large Cell lymphomas) and Hodgkin's lymphoma, leukemia, and multiple myeloma.

“Therapeutic treatment” and “cancer therapies” refers to chemotherapy, hormonal therapy, radiotherapy, and immunotherapy.

The terms “overexpress,” “overexpression” or “overexpressed” interchangeably refer to a protein that is transcribed or translated at a detectably greater level, usually in a cancer cell, in comparison to a normal cell. The term includes overexpression due to transcription, post transcriptional processing, translation, post-translational processing, cellular localization (e.g, organelle, cytoplasm, nucleus, cell surface), and RNA and protein stability, as compared to a normal cell. Overexpression can be detected using conventional techniques for detecting mRNA (i.e., RT-PCR, PCR, hybridization) or proteins (i.e., ELISA, immunohistochemical techniques, mass spectroscopy). Overexpression can be 10%, 20%, 30%, 40%, 50%, 60%, 70%, 80%, 90% or more in comparison to a normal cell. In certain instances, overexpression is 1-fold, 2-fold, 3-fold, 4-fold or more higher levels of transcription or translation in comparison to a normal cell.

The terms “cancer-associated antigen” or “tumor-specific marker” or “tumor marker” interchangeably refers to a molecule (typically protein or nucleic acid such as RNA) that is expressed in the cell, expressed on the surface of a cancer cell or secreted by a cancer cell in comparison to a normal cell, and which is useful for the diagnosis of cancer, for providing a prognosis, and for preferential targeting of a pharmacological agent to the cancer cell. Oftentimes, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. Oftentimes, a cancer-associated antigen is a cell surface molecule that is inappropriately synthesized in the cancer cell, for instance, a molecule that contains deletions, additions or mutations in comparison to the molecule expressed on a normal cell. Oftentimes, a cancer-associated antigen will be expressed exclusively on the cell surface of a cancer cell and not synthesized or expressed on the surface of a normal cell. Exemplified cell surface tumor markers include the proteins c-erbB-2 and human epidermal growth factor receptor (HER) for breast cancer, PSMA for prostate cancer, and carbohydrate mucins in numerous cancers, including breast, ovarian and colorectal.

It will be understood by the skilled artisan that markers may be used singly or in combination with other markers for any of the uses, e.g., diagnosis or prognosis of melanoma, disclosed herein.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, Mouse; rabbit; or a bird; reptile; or fish.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of cancer antigens. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing finctionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al, Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al, Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Diagnostic and Prognostic Methods

The present invention provides methods of diagnosing a cancer by examining cancer antigens (either the protein or the RNA encoding the protein) such as VEGF, CEA, and EGF, or a combination thereof in saliva samples, including wild-type, truncated or alternatively spliced forms. Diagnosis involves determining the level of a polypeptide or polynucleotide of the invention in a patient and then comparing the level to a baseline or range. Typically, the baseline value is representative of a polypeptide or polynucleotide of the invention in a healthy person not suffering from cancer, as measured using saliva or other biological sample such a serum or blood. Variation of levels of a polypeptide or polynucleotide of the invention from the baseline range (either up or down) indicates that the patient has a cancer or is at risk of developing a cancer.

As used herein, the term “providing a prognosis” refers to providing a prediction of the probable course and outcome of a cancer such as breast cancer, including prediction of metastasis, disease free survival, overall survival, etc. The methods can also be used to devise a suitable therapy for cancer treatment, e.g., by indicating whether or not the cancer is still at an early stage or if the cancer had advanced to a stage where aggressive therapy would be ineffective.

Antibody reagents can be used in assays to detect expression levels of VEGF, CEA, and EGF in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif.; Kit #449430) and can be performed using a Behring Nephelometer Analyzer (Fink et al., J. Clin. Chem. Clin. Biochem., 27:261-276 (1989)).

Specific immunological binding of the antibody to nucleic acids can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the nucleic acid is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), in the physical form of sticks, sponges, papers, wells, and the like. An assay strip can be prepared by coating the antibody or a plurality of antibodies in an array on a solid support. This strip can then be dipped into the test sample and processed quickly through washes and detection steps to generate a measurable signal, such as a colored spot.

Alternatively, nucleic acid binding molecules such as probes, oligonucleotides, oligonucleotide arrays, and primers can be used in assays to detect differential RNA expression of VEGF, CEA, and EGF in patient samples, e.g., RT-PCR. In one embodiment, RT-PCR is used according to standard methods known in the art. In another embodiment, PCR assays such as Taqman® assays available from, e.g., Applied Biosystems, can be used to detect nucleic acids and variants thereof. In other embodiments, qPCR and nucleic acid microarrays can be used to detect nucleic acids. Reagents that bind to selected cancer biomarkers can be prepared according to methods known to those of skill in the art or purchased commercially.

Analysis of nucleic acids can be achieved using routine techniques such as Southern analysis, reverse-transcriptase polymerase chain reaction (RT-PCR), or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al. and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Analysis of nucleic acid markers and their variants can be performed using techniques known in the art including, without limitation, microarrays, polymerase chain reaction (PCR)-based analysis, sequence analysis, and electrophoretic analysis. A non-limiting example of a PCR-based analysis includes a Taqman® allelic discrimination assay available from Applied Biosystems. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nat. Biotechnol., 16:381-384 (1998)), and sequencing by hybridization. Chee et al., Science, 274:610-614 (1996); Drmanac et al., Science, 260:1649-1652 (1993); Drmanac et al., Nat. Biotechnol., 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Other methods for detecting nucleic acid variants include, e.g., the INVADER® assay from Third Wave Technologies, Inc., restriction fragment length polymorphism (RFLP) analysis, allele-specific oligonucleotide hybridization, a heteroduplex mobility assay, single strand conformational polymorphism (SSCP) analysis, single-nucleotide primer extension (SNUPE) and pyrosequencing.

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different markers. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection. Other useful physical formats include sticks, wells, sponges, and the like.

Analysis can be carried out in a variety of physical formats. For example, the use of microtiter plates or automation could be used to facilitate the processing of large numbers of test samples. Alternatively, single sample formats could be developed to facilitate diagnosis or prognosis in a timely fashion.

Alternatively, the antibodies or nucleic acid probes of the invention can be applied to patient samples immobilized on microscope slides. The resulting antibody staining or in situ hybridization pattern can be visualized using any one of a variety of light or fluorescent microscopic methods known in the art.

Analysis of the protein or nucleic acid can also be achieved, for example, by high pressure liquid chromatography (HPLC), alone or in combination with mass spectrometry (e.g., MALDI/MS, MALDI-TOF/MS, tandem MS, etc.).

Compositions, Kits and Integrated Systems

The invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize VEGF, CEA, and EGF.

### EXAMPLES

The following examples are offered to illustrate, but not to limit the claimed invention.

### Example 1

### Salivary Protein Factors are Elevated in Breast Cancer Patients

Patients and Methods

Subjects

Subject recruitment and sample collection were within the guidelines of the Institutional Review Board at the University of California Los Angeles Medical Center. The inclusion criteria for the cancer group were as follows: 1) capable of giving informed consent; 2) not pregnant or lactating; 3) no active oral/dental disease; 4) no prior (within 2 years) or concurrent non-breast malignancies, except for non-melanomatous skin cancers, carcinoma in situ of the cervix, or benign tumors such as adenomas; and 5) a diagnosis of breast cancer. These patients were enrolled prior to definitive surgery that would excise the tumor. The control subjects were healthy volunteers recruited from the both the dental and medical centers at UCLA.

Saliva Collection

Unstimulated whole saliva samples were collected with previously established protocols (7-8). Subjects were asked to refrain from eating, drinking, smoking, or oral hygiene procedures for at least 30 minutes before the collection. Lipstick was wiped off, and the subject rinsed her mouth once with plain water. Typically, patients donated approximately 5-10 ml of saliva. Samples were then centrifuged at 2,600 g for 15 minutes at 4° C. The supernatant was then stored at −80° C. until use. Of note, protease inhibitors cocktail, containing 1 ul aprotinin, 10 ul PMSF (phenylmethanesulfonyl fluoride) and 3 ul sodium orthovanadate (all from Sigma, St. Louis, Mo.) were added to each 1 ml saliva sample.

ELISA Analysis

Measurement of salivary proteins was performed by enzyme linked immunosorbent assay (ELISA) according to the manufacturers' instructions. ELISA's for VEGF and EGF were purchased from R&D (Minneapolis, Minn.), and CEA from Biomeda Corp. (Foster City, Calif.). The minimum detectable levels were as follows: 9 pg/ml for VEGF, 0.7 pg/ml for EGF, and 1.5 ng/ml for CEA. The dilutions were as follows: 1:8 for VEGF, 1:25 for EGF, and 1:4 for CEA, using the sample diluents provided. The signals were read on a BIOTEK microplate reader (Winooski, Vt.).

Data Analysis

To assess the association of the level of peptide expression with breast cancer, the means and standard deviations of salivary factors were calculated separately for each group of subjects and the simple group comparison analysis was performed by the Wilcoxon test. Multiple regression analysis was also performed to consider the potential effect of age and ethnicity, (results not shown), such as the regression model on each of 3 peptides were constructed with age, ethnicity, cancer/normal group as independent variables.

To evaluate the predictive power of each of the peptides, the receiver operating characteristic (ROC) curve analysis was conducted on the simple logistic models with peptide expression as an independent variable and cancer/control group as a dependent variable. The ROC curve analysis was repeated on the best model selected from the stepwise selection method that tested the inclusion of various protein combinations in the logistic regression model. Then, the area under the curve (AUC) was computed via numerical integration of the ROC curves. The factor or combination of factors that has the largest AUC was identified as having the strongest predictive power of detecting breast cancer. The test of salivary growth factor was also evaluated using measures such as sensitivity (the proportion of people detected who have the disease) and specificity (the proportion of people who do not have the disease regarded as negative).

Results

Subject Characteristics

All subjects were female. Table 1 summarizes the characteristics for the 49 control subjects and 49 breast cancer patients. There was no significant difference between the healthy controls and cancer subjects in regards to tobacco use, diabetes, hepatitis, and HIV status. The mean age of controls was lower than that of breast cancer patients (41.4±12.4 years versus 54.8±11.2 years, p<0.0001). Race was also a significant factor (p=0.0116). In the cancer group, four had only stage 0 (DCIS-ductal carcinoma in situ). Among the 42 invasive cancer cases, all except three had final pathologic staging as follows: one local recurrence, fourteen stage 1, fourteen stage 2, seven stage 3 (one of whom had only residual DCIS at the time of saliva collection after neoadjuvant chemotherapy), and three stage 4.

Salivary Protein Levels

Salivary fluid levels for VEGF, EGF and CEA in breast cancer and control groups are displayed in Table 2. The Wilcoxon test shows that there is a significant difference between breast cancer patients and the control group in their levels of each of these three salivary proteins' expression. Multiple regression analysis also shows consistent result, such that after adjusting age and ethnicity effect, significant positive associations between each of these three salivary proteins and cancer remain.

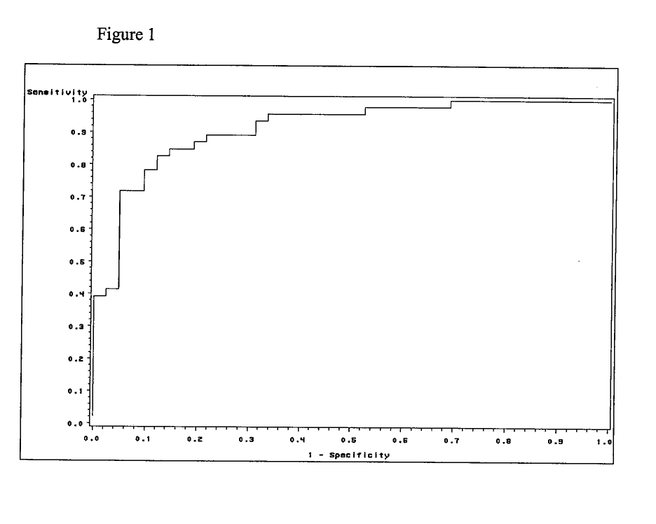

To evaluate the predictive power of each salivary protein individually and in combination, we first found the best logistic regression model by the stepwise model selection methods. This method revealed that the logistic model including the VEGF and EGF proteins together fit the data best. Then ROC analysis was performed on logistic models with each protein separately along with the best combination in Table 3. The AUC's for VEGF, EGF, and CEA were 80%, 77%, and 65%, respectively. The sensitivity and specificity were as follows: 74% and 73% for VEGF, 78% and 68% for EGF, and 70% and 56% for CEA. The best combination was salivary VEGF plus EGF with 83% sensitivity, 74% specificity and AUC 84%. The corresponding ROC curve is displayed as FIG. 1.

Discussion

We report here for the first time that VEGF and CEA levels are significantly increased in the saliva of breast cancer patients, in comparison with healthy control subjects. The most potent angiogenic factor VEGF has previously been detected in saliva of healthy individuals (9-10). We also observed elevated EGF levels, which is consistent with a publication from the Navarro group in Spain (11). In the United States, the Streckfus group has reported that Her-2 and CA15-3 levels are elevated in cancer versus control subjects' saliva (12-13). Another study of 25 breast cancer patients showed that salivary Her-2 exhibit a significant difference between the pre and post therapy values (14).

The finding of elevated angiogenic factors in the saliva is consistent with the fact that the process of angiogenesis, i.e. the formation of new blood vessels, plays a critical role in breast tumor growth and metastasis (15). Since many angiogenic factors have been identified and sequenced, we were among the first to ask whether the level of any of these factors could be detected in bodily fluids, and whether their levels would have any clinical relevance in cancer diagnostics and monitoring (16-17). These angiogenic molecules are either released by the tumor cells themselves (18), or mobilized from extracellular matrix and/or released by host cells such as macrophages recruited into the tumor. Studies from our laboratory and from other institutions have shown that angiogenic factors can be significantly elevated in the serum and urine of breast cancer patients. The levels of certain angiogenic factors have been shown to correlate with the disease stage of the tumor (19).

Our study constitutes a phase II validation study within the guidelines set forth by the NCI Early Detection Research Network (EDRN) (20). The next step is to conduct a phase III blinded detection trial with a large number of new cases of breast cancer and control subjects to determine the robustness of VEGF, EGF and CEA to predict and discriminate saliva from controls versus breast cancer patients.

TABLE 1
Subject characteristics

| | Healthy | Cancer | |
|---|---|---|---|
| | controls | subjects | p |
| Number | 49 | 49 | |
| Age mean ± SD (year) | 41.4 ± 12.4 | 54.8 ± 11.2 | <0.0001^{a} |
| Race | | | 0.0116^{b} |
| White (including Hispanic) | 25 (51%) | 39 (80%) | |
| Black | 11 (22%) | 4 (8%) | |
| Asian | 13 (27%) | 6 (12%) | |
| Tobacco use | | | |
| Diabetes | | | |
| Hepatitis | | | |
| HIV | | | |

^{a}Wilcoson test,
^{b}Chi-square test.

TABLE 2
Wilcoxon Test for each salivary peptide (Mean ± SD)

| | Healthy controls | Cancer subjects | p |
|---|---|---|---|
| VEGF | 2.1 ± 1.2 | 3.7 ± 1.6 | <.0001 |
| EGF | 2.1 ± 1.3 | 3.7 ± 1.7 | <.0001 |
| CEA | 66.1 ± 27.1 | 83.0 ± 31.0 | 0.0106 |

TABLE 3
ROC curve analysis

| | AUC (%) | Sensitivity (%) | Specificity (%) |
|---|---|---|---|
| VEGF | 80 | 74 | 73 |
| EGF | 77 | 78 | 68 |
| CEA | 65 | 70 | 56 |
| VEGF + EGF | 84 | 83 | 74 |

### REFERENCES

1) Wood W C, Muss H B, Solin L J, Olopade O I. Malignant tumors of the breast. In: DeVita V T, Hellman S, Rosenbert S A, editors. Cancer: principles & practice of oncology. Philadelphia: Lippincott Williams & Wilkins; 2005. p. 1415-77.

2) Cheung K L, Graves C R, Robertson J F. Tumour marker measurements in the diagnosis and monitoring of breast cancer. Cancer Treat Rev 2000; 26:91-102.

3) Posadas E M, Simpkins F, Liotta L A, MacDonald C, Kohn E C. Proteomic analysis for the early detection and rational treatment of cancer- realistic hope? Ann Oncol 2005;16:16-22.

4) Anker P, Mulcahy H, Stroun M. Circulating nucleic acids in plasma and serum as a noninvasive investigation for cancer: time for large-scale clinical studies? Int J Cancer 2003;103: 149-52.

5) Kabbinavar F, Hurwitz H I, Fehrenbacher L, et al. Phase II, randomized trial comparing bevacizumab plus fluorouracil (FU)/leucovorin (LV) with FU/LV alone in patients with metastatic colorectal cancer. J Clin Oncol 2003;21:60-5.

6) Byrne B J, Garst J. Epidermal growth factor receptor inhibitors and their role in non-small-cell lung cancer. Curr Oncol Rep 2005;7:241-7.

7) Li Y, St. John MAR, Zhou X, et al. Salivary transcriptome diagnostics for oral cancer detection. Clin Cancer Res 2004;10:8442-50.

8) Navazesh M. Methods for collecting saliva. Ann N Y Acad Sci 1993;694:72-7.

9) Pammer J, Weninger W, Mildner M, Burian M, Wojta J, Tschachler E. Vascular endothelial growth factor is constitutively expressed in normal human salivary glands and is secreted in the saliva of healthy individuals. J Path 1998; 186:186-91.

10) Taichman N S, Cruchley A T, Fletcher L M, et al. Vascular endothelial growth factor in normal human salivary glands and saliva: a possible role in the maintenance of mucosal homeostasis. Lab Invest 1998;78:869-75.

11) Navarro M A, Mesia R, Diez-Gilbert O, Rueda An, Ojeda B, Alonso M C. Epidermal growth factor in plasma and saliva of patients with active breast cancer and breast cancer patients in follow-up compared with healthy women. Breast Cancer Res Treatm 1997;42:83-6.

12) Streckfus C, Bigler L, Tucci M, Thigpen J T. A preliminary study of CA15-3, c-erbB-2, epidermal growth factor receptor, cathepsin-D, and p53 in saliva among women with breast carcinoma. Cancer Inv 2000;18:101-9.

13) Streckfus C, Bigler L, Dellinger T, Dai X, Kingman A, Thigpen J T. The presence of soluble c-erbB-2 in saliva and serum among women with breast carcinoma: A preliminary study. Clin Cancer Res 2000;6:1363-70.

14) Bigler L R, Streckus C F, Copeland L, et al. The potential use of saliva to detect recurrence of disease in women with breast carcinoma. J Oral Pathol Med 2002;31:421-31.

15) Folkman J. Role of angiogenesis in tumor growth and metastasis. Semin Oncol 2002;29: 15-8.

16) Nguyen M, Watanabe H, Budson A, Richie J, Hayes D, Folkman J. Elevated levels of an angiogenic peptide, basic fibroblast growth factor, in the urine of patients with a wide spectrum of cancers. J Natl Cancer Inst 1994;86:356-61.

17) Liu Y, Wang J L, Chang H, Barsky S H, Nguyen M. Breast cancer diagnosis with nipple fluid bFGF. Lancet 2000;356:567.

18) Soutter A, Nguyen M, Watanabe H, Folkman J. Basic fibroblast growth factor secreted by an animal tumor is detectable in urine. Cancer Res 1993;53:5297-9.

19) Nguyen M. Angiogenic factors as tumor markers. Invest New Drug 1997;15:29-37.

20) Pepe M S, Etzioni R, Feng Z, et al. Phases of biomarker development for early detection of cancer. J Natl Cancer Inst 2001;93:1054-61.

21) PCT/US2005/005263

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of diagnosing breast cancer in a subject, the method comprising the steps of:
(a) analyzing a saliva sample from the subject with an assay that specifically detects a marker selected from the group consisting of VEGF, CEA, and EGF nucleic acid and VEGF, CEA, and EGF polypeptide; and
(b) determining whether or not the marker is overexpressed in the sample;
thereby providing a diagnosis for breast cancer.

2. The method of claim 1, wherein the assay detects protein and is ELISA, Western blotting, flow cytometry, immunofluorescence, immunohistochemistry, or mass spectroscopy.

3. The method of claim 1, wherein the assay detects nucleic acid and is mass spectroscopy, PCR, microarray hybridization, thermal cycle sequencing, capillary array sequencing, or solid phase sequencing.

4. The method of claim 1, wherein the assay comprises a reagent that binds to a protein.

5. The method of claim 4, wherein the reagent is an antibody.

6. The method of claim 5, wherein the reagent is a monoclonal antibody.

7. The method of claim 1, wherein the assay comprises a reagent that binds to a nucleic acid.

8. The method of claim 7, wherein the reagent is a nucleic acid.

9. The method of claim 8, wherein the reagent is an oligonucleotide.

10. The method of claim 9, wherein the reagent is an RT-PCR primer set.

11. The method of claim 4, wherein the assay is ELISA, Western blotting, flow cytometry, immunofluorescence, immunohistochemistry, mass spectroscopy.

12. The method of claim 1, comprising the step of analyzing a saliva sample from the subject with an assay that specifically detects VEGF and EGF and determining whether or not VEGF and EGF protein are overexpressed in the sample.

13. The method of claim 1, wherein the marker is VEGF protein or RNA.

14. The method of claim 1, wherein the marker is EGF protein or RNA.

15. The method of claim 1, wherein the marker is CEA protein or RNA.

16. A method of providing a prognosis for a breast cancer, the method comprising the steps of
(a) analyzing a saliva sample from the subject with an assay that specifically detects a marker selected from the group consisting of VEGF, CEA, and EGF nucleic acid and VEGF, CEA, and EGF polypeptide; and
(b) determining whether or not the marker is overexpressed in the sample;
thereby providing a prognosis for breast cancer.

17. The method of claim 16, wherein the assay detects protein and is ELISA, Western blotting, flow cytometry, immunofluorescence, immunohistochemistry, or mass spectroscopy.

18. The method of claim 16, wherein the assay detects nucleic acid and is mass spectroscopy, PCR, microarray hybridization, thermal cycle sequencing, capillary array sequencing, or solid phase sequencing.

19. The method of claim 16, wherein the assay comprises a reagent that binds a protein.

20. The method of claim 19, wherein the reagent is an antibody.

21. The method of claim 20, wherein the reagent is a monoclonal antibody.

22. The method of claim 16, wherein the reagent binds a nucleic acid.

23. The method of claim 22, wherein the reagent is a nucleic acid.

24. The method of claim 23, wherein the reagent is an oligonucleotide.

25. The method of claim 24, wherein the reagent is an RT-PCR primer set.

26. The method of claim 20, wherein ELISA is used to determine whether or not VEGF, EGF, or CEA protein is expressed in the saliva sample.

27. The method of claim 16, comprising the step of analyzing a saliva sample from the subject with an assay that specifically detects VEGF and EGF and determining whether or not VEGF and EGF protein are overexpressed in the sample.

28. The method of claim 16, wherein the marker is VEGF protein or RNA.

29. The method of claim 16, wherein the marker is EGF protein or RNA.

30. The method of claim 16, wherein the marker is CEA protein or RNA.

31. A method of monitoring the efficacy of a treatment for a breast cancer, the method comprising the steps of
(a) analyzing a saliva sample from the subject with an assay that specifically detects a marker selected from the group consisting of VEGF, CEA, and EGF nucleic acid and VEGF, CEA, and EGF polypeptide; and
(b) determining whether or not the marker is overexpressed in the sample;
thereby monitoring the efficacy of a treatment for breast cancer.
